(19) United States
(12) Patent Application Publication
(10) Pub. No.: US 2007/0031861 A1
(43) Pub. Date: Feb. 8, 2007

(54) Methods for determining nucleotide sequence information

(75) Inventor: Xing Su, Cupertino, CA (US)

Correspondence Address: Raj S. Dave;Morrison & Foerster LLP, Suite 300, 1650 Tysons Blvd., McLean, VA, 22102, US

(73) Assignee: INTEL CORPORATION

(21) Appl. No.: 11/414,611

(22) Filed: Apr. 28, 2006

Related U.S. Application Data
(62) Division of application No. 10/748,374, filed on Dec. 29, 2003.

(51) Int. Cl. C12Q 1/68

(52) U.S. Cl. 435/6

(57) ABSTRACT

Provided herein, is a nucleic acid sequencing method based on detection of Raman signatures of oligonucleotide probes. Raman signatures of individually captured nucleic acid probes, optionally labeled by a Raman label or a positively charged enhancer, are detected. The sequences of captured probes are used to identify the nucleotide sequences of captured probes and complementary target nucleic acids, which are then aligned and used to obtain nucleic acid sequence information. In another embodiment, a method is provided for determining a nucleotide occurrence at a target nucleotide position of a target nucleic acid, that utilizes binding of the target nucleic acid to a labeled oligonucleotide probe that binds to the target nucleic acid, wherein the labeled oligonucleotide probe includes a first label and a second label, the first label being capable of affecting an optical property of the second label.

### CROSS REFERENCE TO RELATED APPLICATION(S)

This application is a divisional application of U.S. application Ser. No. 10/748,374 filed Dec. 29, 2003, now pending. The disclosure of the prior application is considered part of and is incorporated by reference in the disclosure of this application.

### BACKGROUND OF THE INVENTION

1. Field of the Invention

The invention relates generally to detection methods, and more specifically to methods for detecting and sequencing biomolecules.

2. Background Information

Genetic information is stored in the form of very long molecules of deoxyribonucleic acid (DNA), organized into chromosomes. These chromosomes include approximately three billion nucleotides, which form the human genome. The sequence of nucleotides in the chromosomes plays a large role in determining the characteristics of each individual. Many common diseases are based at least in part on variations in nucleotide sequences of the human genome between individuals.

Determination of the entire sequence of the human genome has provided a foundation for identifying the genetic basis of such diseases. However, a great deal of experimentation remains to be done to identify the genetic variations associated with each disease. This experimentation requires DNA sequencing of portions of chromosomes in individuals or families exhibiting each such disease, in order to identify specific changes in DNA sequence that promote the disease. Ribonucleic acid (RNA), an intermediary molecule in processing genetic information, can also be sequenced to identify the genetic bases of various diseases.

Current sequencing methods require that many copies of a template nucleic acid of interest be produced, cut into overlapping fragments and sequenced, after which the overlapping DNA sequences are assembled into the complete gene. This process is laborious, expensive, inefficient and time-consuming. It also typically requires the use of fluorescent or radioactive labels, which can potentially pose safety and waste disposal problems. Accordingly, a need exists for improved nucleic acid sequencing methods which are less expensive, more efficient, and safer than present methods.

An understanding of nucleotide sequence variations that lead to various diseases requires techniques for detecting these variations. In particular, techniques for detecting subtle changes in nucleotide sequence have become more important due in part to recent scientific advances in identifying polymorphisms, especially single nucleotide polymorphisms (SNPS). Furthermore, methods for detecting subtle changes in nucleic acids have become more important to translate genetic discoveries into accurate and cost-effective genetic tests. Accordingly, there is a need for a sensitive and simple detection method for genotyping that is able to distinguish target molecules with subtle differences.

Current methods for detecting nucleotide variations, require that different probes are used to distinguish respective alleles of a target gene; or a probe is modified biochemically during an assay. These methods are time consuming and costly. Thus, there is a need for a simple, sensitive, and low cost method for performing genotype analysis.

### BRIEF DESCRIPTION OF THE DRAWINGS

FIG. 1 diagrammatically illustrates a method of the invention.

FIG. 2 illustrates the structure of an exemplary Raman-active oligonucleotide probe.

FIG. 3 illustrates an exemplary biochip design and an exemplary sequence determination method using the biochip.

FIGS. 4A-4D provide a series of Raman spectra and corresponding oligonucleotide structure for a series of oligonucleotide probes with and without a positively charged enhancer. The figure illustrates enhancement of Raman emission intensity by an amine group enhancer.

FIGS. 5A and 5B illustrate examples of synchronous fluorescence scan spectra of probe-target complexes. FIG. 5A diagrammatically illustrates the nucleotide sequence and probe locations of a labeled oligonucleotide probe 510 that includes a FRET pair 560, 570, and illustrates alignment of the labeled oligonucleotide probe 510 to various target nucleic acids 520, 530, 540, 550. FIG. 5B shows fluorescent spectra generated for the various hybridization pairs shown in FIG. 5A.

FIG. 6 illustrates a MEMS device for probe-target complex detection using an AC field.

### DETAILED DESCRIPTION OF THE INVENTION

The disclosed methods are based, in part, on the discovery of the advantages of combining Raman spectroscopy with sequencing by hybridization. Raman spectroscopy provides an advantage in that large numbers of Raman-active signal molecules are known (See e.g., “Standard Raman Spectra” (Sadtler Research Laboratories); and “TRC spectral data. Raman” (Thermodynamics Research Center)) and can be used to label sequencing by hybridization probes. Because a large number of Raman-active signal molecules can be made, sequencing based on signatures of individual molecules is possible. Furthermore, the disclosed methods are based in part on the discovery of a novel Raman enhancer, which makes it possible to detect oligonucleotides that are otherwise undetectable by Raman spectroscopy, or produce very low intensity Raman emissions.

The methods for nucleic acid sequencing and for identifying a nucleotide occurrence at a target nucleic acid position provided herein, can be performed more quickly than traditional sequencing methods because they involve several reaction steps fewer than traditional methods, and because they can be performed in a highly parallel, manner in micro or nano scales. Therefore, a relatively large amount of sequence information can be obtained in a relatively short time. In addition, the methods disclosed herein are low cost, since they eliminate expensive reagents used in target molecule amplification and chemical labeling using traditional methods.

Accordingly, in one embodiment a nucleic acid sequencing method based on detection of Raman signatures of oligonucleotide probes in a sequencing by hybridization reaction, is provided. Raman signatures of individually captured nucleic acid probes labeled by Raman labels are detected. The sequences of captured probes are used to identify the nucleotide sequences of captured probes and complementary target nucleic acids, which are then aligned and used to obtain nucleic acid sequence information. The method can be used, for example, for large scale genome sequencing, detection of nucleotide occurrences at single nucleotide polymorphisms (SNPs), sequence comparison, genotyping, disease correlation and drug development.

In another embodiment, a method for determining a nucleotide sequence of a target nucleic acid is provided, that includes contacting the nucleic acid, or a fragment thereof, with a population of capture oligonucleotide probes bound to a substrate at a series of spot locations, to form probe-target duplex polynucleotides comprising single-stranded overhangs, contacting the probe-target duplex nucleic acids with a population of Raman-active oligonucleotide probes to allow binding of the Raman-active oligonucleotide probes to the single-stranded overhangs, wherein each Raman-active oligonucleotide probe generates a distinct Raman signature, and detecting Raman-active oligonucleotide probes that bind the template nucleic acid using Raman spectroscopy, thereby determining a nucleotide sequence of the target nucleic acid. Furthermore, the location of the spot for each of the captured Raman-active oligonucleotide probes can be identified and used to determine the nucleotide sequence of the target nucleic acid.

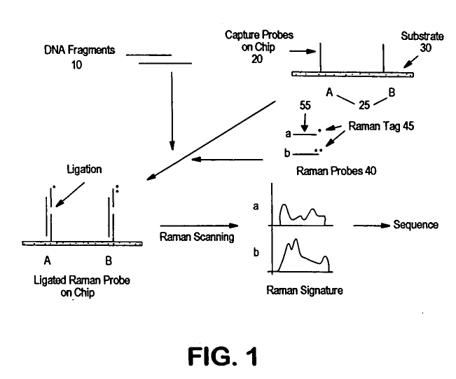

Methods of these embodiment of the invention are sometimes referred to herein as sequencing by hybridization methods. As shown in FIG. 1, two types of probes are typically employed in methods of this embodiments and used to probe a target nucleic acid molecule, or a fragment thereof 10: a) probes immobilized on a substrate 20, such as a biochip (i.e. capture oligonucleotide probes 20); and b) Raman-active oligonucleotide probes 40. As discussed in more detail herein, capture probes 20 are nucleic acid molecules with known nucleotide sequences. These probes are synthesized by standard chemical methods and are not required to be labeled. They are typically immobilized on a solid surface 30 at either their 5′ or 3′ end. Standard chemical cross linking techniques can be used for probe immobilization, such as thiol-gold linkage or amine-aldehyde linkage, as disclosed in more detail herein.

A Raman-active oligonucleotide probe 40 includes a synthetic nucleic acid with a known nucleotide sequence and optionally one or more Raman labels 45 or one or more positively charged enhancers. Raman labels 45 are chemical compounds with detectable and unique Raman signatures, as disclosed in more detail herein. They can be covalently attached to nucleic acid sequences. An enhancer is a compound or a moiety of a compound that stimulates Raman activity of an oligonucleotide.

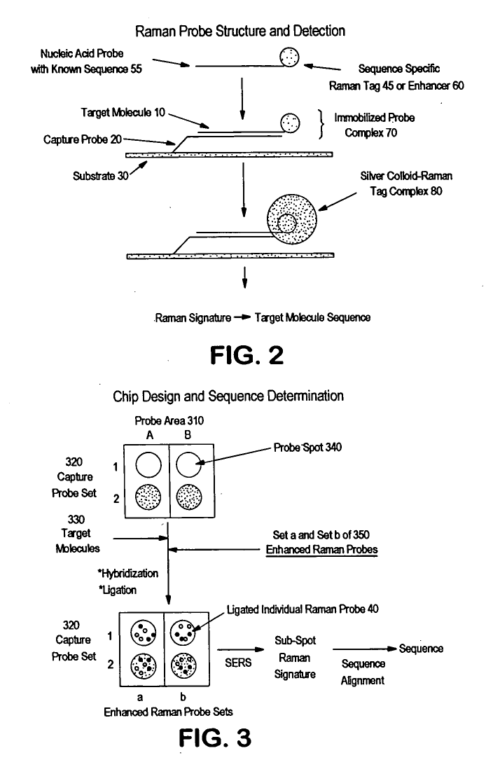

When a Raman-active oligonucleotide probe 40 is captured on a surface 30 by a target sequence-dependent reaction, the presence of a complementary sequence to the Raman-active oligonucleotide probe can be determined by detecting the presence of the corresponding Raman label (FIG. 2). The capturing step is an immobilization process, which can be done through sequence specific hybridization or ligation. If the complement of the target sequence is not present on the target nucleic acid, the target nucleic acid, and therefore, the Raman-active oligonucleotide probes are not be immobilized.

As discussed above, a population of Raman-active oligonucleotide probes are provided herein. Each Raman-active oligonucleotide probe is capable of generating a detectable Raman signal. The Raman signal for at least some of the oligonucleotide probes in the population of Raman-active oligonucleotide probes can be generated intrinsically. An oligonucleotide “intrinsically generates” a Raman signal when a detectable Raman signal is detected from the oligonucleotide without covalent attachment of a label or a positively-charged enhancer. Whether an oligonucleotide intrinsically generates a Raman signal will depend on the sensitivity of the Raman detector used to detect the signal. As illustrated in the Examples herein, oligonucleotides that include purine residues, especially adenosine residues, as well as adenosine derivatives such as, for example, 8-aza-adenine or dimethyl-allyl-amino-adenine are more likely to intrinsically generate a detectable Raman signal.

Oligonucleotides that do not intrinsically generate a Raman signal, or that generate a weak Raman signal, in some aspects, are covalently bound to a positively charged enhancer. Accordingly, disclosed herein are populations of oligonucleotides, at least some of which are covalently attached to a positively charged enhancer. As illustrated in the Examples herein, for example, oligonucleotides with a high percentage of pyrimidine nucleotides can have weak or undetectable intrinsic Raman activity. These oligonucleotides can be covalently bound to a positively charged group to render the Raman signal detectable or to increase the intrinsic signal generated by the oligonucleotide, as illustrated in the Examples herein. Not to be limited by theory, it is believed that the positively charged groups increases the association and orientation of the oligonucleotide with a SERS surface. Alternatively, a pyrimidine-rich oligo nucleotide can be hybridized, completely or partially, to an adenosine-rich oligonucleotides to obtain an enhanced Raman signal.

For example, oligonucleotides that contain less than 5, 4, 3, 2, or 1 purine residue can be covalently bound to a positively charged enhancer or hybridized to a purine or adenosine-rich oligonucleotide. In other examples, oligonucleotides that contain less than 5, 4, 3, 2, or 1 adenosine residues are covalently bound to a positively charged enhancer of the present invention or hybridized to a purine or adenosine-rich oligonucleotide.

Accordingly, in another embodiment, a method for detecting a nucleic acid is provided, that includes irradiating the nucleic acid with light, wherein the nucleic acid comprises a positively-charged enhancer; and detecting a Raman signal generated by the irradiated nucleic acid. In certain aspects, the positively charged enhancer is an amine group. In certain examples, the nucleic acid does not generate a detectable signal in the absence of the positively-charged enhancer. In certain aspects, the nucleic acid is composed of less than 5, 4, 3, 2, or 1 purine residue, and/or less than 10%, 5%, or 1% purine residues. In certain aspects, the nucleic acid includes no purine residues.

Positively charged enhancers of the present invention include any positively charged group that can be attached to an oligonucleotide without blocking binding of the oligonucleotide to a complementary sequence. In general, any group that contains a heteroatom (i.e., N, O, S, P) can bear a positive charge. For example, an amine with a positive charge becomes an ammonium group, a hydroxyl (—OH) becomes a hydronium, a thiol (—SH) becomes SH²⁺. Methods of attaching these positively charged enhancer moieties to an oligonucleotide are readily available. In one non-limiting example, the enhancer can be a primary amine with an alkyl or aryl chain of 1, 2, 3, 4, 5, 6, 7, 8, 9, 10, 15, 20, or 25 carbon atoms. The positively charged group can be attached to any reactive group on an oligonucleotide that contains a modified base or spacer. Introducing a spacer into a oligo with a reactive amino group is commercially available (i.e., Qiagen-Operon). Modified bases can be amine-containing bases, such as 8-aza-adenine, zeatin, kinetin, N6-benzoyladenine, 4 pyridine carboxal doxime, dimethyl-allyl-amino-adenine; a modified base can also be thiol-containing bases, such as, 4-amino-6-mercaptopyrazolo[3,4-d]pyrimidine, 2 mercapto-benzimidazole, 8-mercaptoadenine, 6-mercaptopurine. All these bases can be further modified to possess a positive group by chemical attachment through the reactive amine group or the thiol group. All compounds are available from a chemical vendor (Sigma-Aldrich, St. Louis, Mo.). The chemistry for converting a reactive group to positive group is known in the art. Furthermore, as illustrated in the Examples herein, the positively charged enhancer can be attached within an oligonucleotide or at the 5′ or 3′ end of an oligonucleotide. It will be understood that the positively charged group can be attached using any suitable method.

The positively charged group can be singly or multiply positively charged. In certain aspects, the positively charged group is an amino (or ammonium) group, for example a quaternary ammonium label (See e.g., U.S. Pat. No. 6,268,129). Suitably, the amino (or ammonium) group is added to a synthetic oligonucleotide either by a 5′-NH₂ link or by attaching a base containing an aliphatic NH₂ group to the 3′ end of the oligonucleotide, for example by using the enzyme terminal transferase or directly by using a base for termination in a Sanger chain termination protocol which has an aliphatic NH₂ group by which the positively charged group can be added. The positively charged group (which is preferably a quaternary ammonium-containing compound) is then attached to the aliphatic—NH₂ group in the nucleic acid. Conveniently, the quaternary ammonium-containing compound includes hydroxysuccinimidyl ester which reacts with aliphatic—NH₂ groups. The compound can be, for example, trimethyl ammonium hexyryl-N-hydroxysuccinimidyl ester (C5-NHS).

In certain aspects, the population of Raman-active oligonucleotide probes each include a Raman label. A variety of Raman labels are known in the art and can be used with the present invention, provided that they can be attached to an oligonucleotide and that they do not inhibit hybridization of the oligonucleotide to a complementary sequence.

Non-limiting examples of Raman labels, also referred to herein as Raman signal molecules, that can be attached to oligonucleotide probes disclosed herein include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine and aminoacridine. Furthermore, the Raman-active labels can include those that have been identified for use in gene probes (See e.g., Graham et al., Chem. Phys. Chem., 2001; Isola et al., Anal. Chem., 1998). In one aspect, the Raman-active labels include those disclosed in Kneipp et al., Chem Reviews 99, 2957 (1999). These and other Raman labels can be obtained from commercial sources (e.g., Molecular Probes, Eugene, Oreg.).

Raman active labels, in certain aspects include composite organic-inorganic nanoparticles (See U.S. Pat. App. No. 10/748,336, filed Dec. 29, 2003, entitled “Composite Organic-Inorganic Nanoparticles” (referred to herein as COIN nanoparticles or “COINs”)). In these aspects, either one or both the capture oligonucleotide probes and the Raman-active oligonucleotide probes are associated with COIN nanoparticles and detected using SERS.

COINs are Raman-active probe constructs that include a core and a surface, wherein the core includes a metallic colloid including a first metal and a Raman-active organic compound. The COINs can further include a second metal different from the first metal, wherein the second metal forms a layer overlying the surface of the nanoparticle. The COINs can further include an organic layer overlying the metal layer, which organic layer comprises the probe. Suitable probes for attachment to the surface of the SERS-active nanoparticles for this embodiment include, without limitation, antibodies, antigens, polynucleotides, oligonucleotides, receptors, ligands, and the like. However, for these embodiments, COINs are typically attached to an oligonucleotide probe.

The metal for achieving a suitable SERS signal is inherent in the COIN, and a wide variety of Raman-active organic compounds can be incorporated into the particle. Indeed, a large number of unique Raman signatures can be created by employing nanoparticles containing Raman-active organic compounds of different structures, mixtures, and ratios. Thus, the methods described herein employing COINs are useful for the simultaneous determination of nucleotide sequence information from more than one, and typically more than 10 target nucleic acids. In addition, since many COINs can be incorporated into a single COIN bead nanoparticle, the SERS signal from a single COIN particle is strong relative to SERS signals obtained from Raman-active materials that do not contain the nanoparticles described herein. This situation results in increased sensitivity compared to Raman-techniques that do not utilize COINs.

COINs are readily prepared for use in the invention methods using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs used in the invention methods are prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This simple type of COIN is referred to as type I COIN. Type I COINs can typically be isolated by membrane filtration. In addition, COINs of different sizes can be enriched by centrifugation.

In alternative embodiments, the COINs can include a second metal different from the first metal, wherein the second metal forms a layer overlying the surface of the nanoparticle. To prepare this type of SERS-active nanoparticle, type I COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. This type of COIN is referred to as type II COINs. Type II COINs can be isolated and or enriched in the same manner as type I COINs. Typically, type I and type II COINs are substantially spherical and range in size from about 20 nm to 60 nm. The size of the nanoparticle is selected to be very small with respect to the wavelength of light used to irradiate the COINs during detection.

Typically, organic compounds, such as oligonucleotides, are attached to a layer of a second metal in type II COINs by covalently attaching the organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COIN(s) used in the invention methods can include cores containing magnetic materials, such as, for example, iron oxides, and the like. Magnetic COINs can be handled without centrifugation using commonly available magnetic particle handling systems. Indeed, magnetism can be used as a mechanism for separating biological targets attached to magnetic COIN particles tagged with particular biological probes.

Another type of Raman label is a polycyclic aromatic compounds. Other labels that can be of use include cyanide, thiol, chlorine, bromine, methyl, phosphorus and sulfur. In certain embodiments, carbon nanotubes can be of use as Raman labels. The use of labels in Raman spectroscopy is known (e.g., U.S. Pat. Nos. 5,306,403 and 6,174,677).

Raman active labels can be attached directly to probes or can be attached via various linker compounds. Nucleotides that are covalently attached to Raman labels are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman labels that contain reactive groups designed to covalently react with other molecules, for example nucleotides or amino acids, are commercially available (e.g., Molecular Probes, Eugene, Oreg.)

In certain aspects, Raman labels are deposited on a SERS substrate before being detected by SERS. Methods for depositing Raman signal molecules on substrates are known in the art. A detection means or detection unit, can be designed to detect and/or quantify nucleotides by Raman spectroscopy. Various methods for detection of nucleotides by Raman spectroscopy are known in the art. (See, e.g., U.S. Pat. Nos. 5,306,403; 6,002,471; 6,174,677). However, Raman detection of labeled or unlabeled nucleotides at the single molecule level has not previously been demonstrated. Variations on surface enhanced Raman spectroscopy (SERS) or surface enhanced resonance Raman spectroscopy (SERRS) have been disclosed. In SERS and SERRS, the sensitivity of the Raman detection is enhanced by a factor of 10⁶ or more for molecules adsorbed on roughened metal surfaces, such as silver, gold, platinum, copper or aluminum surfaces.

A non-limiting example of a detection means or detection unit is disclosed in U.S. Pat. No. 6,002,471. In this embodiment, the excitation beam is generated by either a Nd:YAG laser at 532 nm wavelength or a Ti:sapphire laser at 365 nm wavelength. Pulsed laser beams or continuous laser beams can be used. The excitation beam passes through confocal optics and a microscope objective, and is focused onto the reaction chamber. The Raman emission light from the nucleotides is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector. The detector includes an avalanche photodiode interfaced with a computer for counting and digitization of the signal. In certain embodiments, a mesh including silver, gold, platinum, copper or aluminum can be included in the reaction chamber or channel to provide an increased signal due to surface enhanced Raman or surface enhanced Raman resonance. Alternatively, nanoparticles that include a Raman-active metal can be included.

Alternative embodiments of detection means or detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source is a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677). The excitation beam can be spectrally purified with a bandpass filter (Corion) and can be focused on the reaction chamber using a 6X objective lens (Newport, Model L6X). The objective lens can be used to both excite the nucleotides and to collect the Raman signal, by using a holographic beam splitter (Kaiser Optical Systems, Inc., Model HB 647-26N1 8) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as charged injection devices, photodiode arrays or phototransistor arrays.

Any suitable form or configuration of Raman spectroscopy or related techniques known in the art can be used for detection of nucleotides, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

A Raman label can be incorporated into a nucleotide prior to the synthesis of an oligonucleotide probe. For example, internal amino-modifications for covalent attachment at adenine (A) and guanine (G) positions are contemplated. Internal attachment can also be performed at a thymine (T) position using a commercially available phosphoramidite. In some embodiments library segments with a propylamine linker at the A and G positions can be used to attach signal molecules to coded probes. The introduction of an internal aminoalkyl tail allows post-synthetic attachment of the signal molecule. Linkers can be purchased from vendors such as Synthetic Genetics (San Diego, Calif.). In one embodiment of the invention, automatic coupling using the appropriate phosphoramidite derivative of the signal molecule is also contemplated. Such signal molecules can be coupled to the 5′-terminus during oligonucleotide synthesis.

In general, Raman labels will be covalently attached to the probe in such a manner as to minimize steric hindrance with the signal molecules, in order to facilitate coded probe binding to a target molecule, such as hybridization to a nucleic acid. Linkers can be used that provide a degree of flexibility to the coded probe. Homo-or hetero-bifunctional linkers are available from various commercial sources.

The point of attachment to an oligonucleotide base will vary with the base. While attachment at any position is possible, in certain embodiments attachment occurs at positions not involved in hydrogen bonding to the complementary base. Thus, for example, attachment can be to the 5 or 6 positions of pyrimidines such as uridine, cytosine and thymine. For purines such as adenine and guanine, the linkage is can be via the 8 position.

The nucleic acid sequence of the target nucleic acid that is determined using methods provided in this embodiment range from a single nucleotide occurrence at a target nucleotide position to a complete sequence of a target nucleic acid. In certain aspects, the target position is a single nucleotide polymorphism position.

Alternatively, a series of nucleotide occurrences at adjacent positions of a target segment of the target nucleic acid can be determined. The target segment, for example, can be less than or equal to the combined length of the capture oligonucleotide probe and the Raman-active oligonucleotide probe. For example, if a 10 nucleotide capture probe and a 20 nucleotide Raman-active oligonucleotide probe are used, the target segment can be 30 nucleotides or less. As another non-limiting example, the target segment can be less than or equal to the length of the Raman-active oligonucleotide probe. Therefore, if the Raman-active oligonucleotide probe is a 10-mer, the target segment can be 10 bases or less, for example.

In some aspects, the nucleotide sequence of the entire target nucleic acid is determined. This is typically done by aligning detected target sequences using methods known in the art. The target sequences, for example, can be overlapping sequences to expedite the alignment process. In some aspects, fragments of a target nucleic acid sequence are analyzed separately, and then data from these separate analysis is combined to determine the nucleotide sequence of the entire target nucleic acid molecule. Fragments of a target nucleic acid molecule can be obtained using known methods such as endonuclease cleavage. Furthermore, the target nucleic acid, or fragments thereof, are typically employed in sequencing by hybridization methods as single stranded nucleic acids, as will be understood. For example, the target nucleic acid molecule can be denatured before it is contacted with probes.

Raman signatures of the Raman-active oligonucleotide probes are obtained by Raman scanning. For example, surface enhanced Raman spectroscopy (SERS) can be used. SERS Raman scanning is typically performed in micrometer or nanometer scale. Raman spectra of individual Raman probes are typically recorded.

As indicated above, and illustrated in FIG. 2, Raman-active oligonucleotide probes 40 include an oligonucleotide probe 55 with a known sequence, and optionally a sequence specific Raman label 45 (also referred to herein as a Raman tag) or a positively charged enhancer 60. An immobilized probe complex 70 is formed during a method of the present embodiment that includes a capture oligonucleotide probe 20, a bound target molecule 10, and a bound Rarnan-active oligonucleotide probe 40. As illustrated in FIG. 2, silver colloids or other nano-particles can be aggregated with the oligonucleotides and/or Raman labels in the presence of mono-valent salts to form a silver colloid-Raman label complex 80 that is detected by Raman spectroscopy. The metal colloids can be pre-made or synthesized in situ.

SERS scanning of Raman-active probe molecules provides a detection method with high sensitivity. In fact, it has been reported that a single nucleotide can be detected by SERS. Higher Raman activities can be obtained by chemical modification of nucleotides, such as by attaching a positively charged enhancer. Therefore, fewer copies of target nucleic acids, and capture oligonucleotide probes are required for detection, than needed for traditional methods. Accordingly, as few as 1000 or less, 500 or less, 250 or less, 125 or less, 100 or less, 50 or less, 25 or less, 20 or less, 15 or less, 10, 9, 8, 7, 6, 5, 4, 3, 2, or 1 molecule of the Raman-active oligonucleotide probe are detected.

The Raman signatures are translated into nucleic acid sub-sequences corresponding to probes. In certain aspects, the nucleotide sequence of capture oligonucleotide probes are determined by their locations on a substrate and sequences of Raman-active oligonucleotide probes are determined by Raman signatures and in some examples by their position on the substrate as well. Thus, typically the sequence of a target nucleic acid, or a fragment (i.e. sub-sequence) thereof, is determined by both the capture probe sequence and the Raman-active oligonucleotide probe sequence. The complete sequence of a target molecule can be deduced by aligning nucleic acid sub-sequences.

Methods for using the identification of hybridizing oligonucleotides to decode sequence information is known in the art. For example, the cited references related to sequencing by hybridization included herein provide detailed methods for decoding polynucleotide sequence information based on a sequencing by hybridization result. Data collected from multiple nanoparticle readings are used to determine the polynucleotide sequence based on a sequence alignment principle (See e.g., Laser Gene program (DNA Star, Mountain View, Calif.). Bioinformatics companies and government agencies provide necessary tools, services, and other associated tools for data processing to determine DNA sequences.

As indicated above, two populations of probes are typically included in a method for sequencing by hybridization using Raman spectroscopy disclosed herein. The populations of labeled oligonucleotide probes are also referred to herein as a “labeled oligonucleotide libraries.” The population of labeled oligonucleotides are typically hybridization probes that include a known nucleotide sequence portion, also referred to as a probe portion. The populations of oligonucleotide probes include a population of capture oligonucleotide probes and a population of Raman-active oligonucleotide probes.

In certain aspects a population of probes, especially Raman-active oligonucleotide probes, includes oligonucleotides with nucleotide sequences that correspond to every possible permutation less than or equal to the length of the oligonucleotides. Typically all of the nucleotides in a population are of an identical length. For example, the oligonucleotides, in certain aspects are equal to or less than 250 nucleotides, 200 nucleotides, 100 nucleotides, 50 nucleotides, 25 nucleotides, 20 nucleotides, 15 nucleotides, 10 nucleotides, 9 nucleotides, 8 nucleotides, 7 nucleotides, 6 nucleotides, 5 nucleotides, 4 nucleotides, or 3 nucleotides in length. For example, but not intended to be limiting, the oligonucleotide is 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 200, or 250 nucleotides in length. For example, the population of labeled probes includes probes of an identical length of between 2 and 50 nucleotides, or for example an identical length of between 3 and 25 nucleotides in length. For example, the population of labeled oligonucleotide probes can include all possible oligonucleotide probes 3 nucleotides in length.

A population of labeled oligonucleotides in certain aspects, includes at least 10, 20, 30, 40, 50, 100, 200, 250, 500, 1000, 10,000, or more oligonucleotides. For example, the population can include substantially all, or all of the possible nucleotide sequence combination for oligonucleotides of an identical length, as is known for at least some sequencing by hybridization reactions (See e.g., U.S. Pat. No. 5,002,867). Substantially all of the possible nucleotide sequence combinations for a given length includes enough of the possible nucleotide sequences to allow unequivocal detection of a hybridizing target nucleic acid.

For the population of Raman-active oligonucleotides, each labeled probe can generate a unique Raman signal. A unique Raman signal as used herein, is a Raman signal that provides a Raman signature that is distinguishable from other Raman signatures of other Raman labels used in the population.

In one embodiment, a method for detecting the nucleotide occurrence of a target nucleotide position in a template nucleic acid is provided that includes contacting the template nucleic acid with one or more capture oligonucleotide probes that bind to the target nucleic acid immediately 5′ to the target nucleotide position, and two or more labeled oligonucleotide probes that bind to a target region that includes the target nucleotide position at a 3′ nucleotide. These aspects are useful, for example, for determining nucleotide occurrence at a single nucleotide polymorphism (SNP).

In certain aspects, the method of determining a nucleotide sequence further includes an optional ligation reaction. The ligation reaction typically involves ligation of a capture oligonucleotide probe to a Raman-active oligonucleotide probe that binds to an adjacent region of a target nucleic acid. After adjacent oligonucleotides are ligated, oligonucleotides that are not immobilized to the substrate can be removed, for example by elevating the temperature or changing the pH of a reaction to denature nucleic acids. Oligonucleotides that are not immobilized to the substrate either directly or indirectly can be washed away and the immobilized oligonucleotides can be detected using SERS. The ligation and wash steps increase the specificity of the reaction.

Accordingly, as shown in FIG. 1, capture oligonucleotide probes 20 can be immobilized on various spots 25 (A and B in FIG. 1) on a substrate 30. In aspects that include a ligation step, a Raman-active oligonucleotide probe 40 ligates to a capture oligonucleotide probe 20 only when the target nucleic acid 10 includes a target segment that is complementary to both the Raman-active oligonucleotide probe 40 and the capture oligonucleotide probe 20. In this aspect, the nucleotide sequence is determined based on Raman signatures of ligated Raman probes and corresponding positions of capture probes.

Adjacent labeled oligonucleotide probes can be ligated together using known methods (see, e.g., U.S. Pat. Nos. 6,013,456). Primer independent ligation can be accomplished using oligonucleotides of at least 6 to 8 bases in length (Kaczorowski and Szybalski, Gene 179:189-193, 1996; Kotler etal., Proc. Natl. Acad. Sci. USA 90:4241-45, 1993). Methods of ligating oligonucleotide probes that are hybridized to a nucleic acid template are known in the art (U.S. Pat. No. 6,013,456). Enzymatic ligation of adjacent oligonucleotide probes can utilize a DNA ligase, such as T4, T7 or Taq ligase or E. coli DNA ligase. Methods of enzymatic ligation are known (e.g., Sambrook et al., 1989).

The nucleic acid sequencing methods provided herein, are sequencing by hybridization reactions, as are known in the art. One or more oligonucleotide probes of known sequence is allowed to hybridize to a target nucleic acid sequence. Binding of the labeled oligonucleotide to the target indicates the presence of a complementary sequence in the target strand. Multiple labeled probes can be allowed to hybridize simultaneously to the target molecule and detected simultaneously. In alternative embodiments, bound probes may be identified attached to individual target molecules, or alternatively multiple copies of a specific target molecule may be allowed to bind simultaneously to overlapping sets of probe sequences. Individual molecules may be scanned, for example, using known molecular combing techniques coupled to a detection mode. (See, e.g., Bensimon et al., Phys. Rev. Lett. 74:4754-57, 1995; Michalet et al., Science 277:1518-23, 1997; U.S. Pat. Nos. 5,840,862; 6,054,327; 6,225,055; 6,248,537; 6,265,153; 6,303,296 and 6,344,319.)

It is unlikely that a given target nucleic acid will hybridize to contiguous probe sequences that completely cover the target sequence. Rather, multiple copies of a target may be hybridized to pools of labeled oligonucleotides and partial sequence data collected from each. The partial sequences may be compiled into a complete target nucleic acid sequence using publicly available shotgun sequence compilation programs. Partial sequences may also be compiled from populations of a target molecule that are allowed to bind simultaneously to a library of labeled oligonucleotide probes, for example in a solution phase.

The substrate on which the capture probe is immobilized can be a polymer, a plastic, a resin, a polysaccharide, a silica or silica-based material, a carbon, a metal, an inorganic glass, a membrane. For example, the substrate can be metal, glass, or plastic. In one aspect, the surface is optically transparent and has surface Si—OH functionalities, such as those found on silica surfaces.

Methods and apparatus for attachment to surfaces and alignment of molecules, such as oligonucleotide probes are known in the art (See, e.g., Bensimon et al., Phys. Rev. Lett. 74:4754-57, 1995; Michalet et al., Science 277:1518-23, 1997; U.S. Pat. Nos. 5,840,862; 6,054,327; 6,225,055; 6,248,537; 6,265,153; 6,303,296 and 6,344,319). Non-limiting examples of surfaces include glass, functionalized glass, ceramic, plastic, polystyrene, polypropylene, polyethylene, polycarbonate, PTFE (polytetrafluoroethylene), PVP (polyvinylpyrrolidone), germanium, silicon, quartz, gallium arsenide, gold, silver, nylon, nitrocellulose or any other material known in the art that is capable of having capture probes attached to the surface. Attachment can be either by covalent or noncovalent interaction. Although in certain embodiments of the invention the surface is in the form of a glass slide or cover slip, the shape of the surface is not limiting and the surface can be in any shape. In some aspects of the invention, the surface is planar.

Nucleic acid immobilization is typically used herein to immobilize capture probes onto a substrate. Immobilization of nucleic acids can be achieved by a variety of methods known in the art. For example, immobilization can be achieved by coating a substrate with streptavidin or avidin and the subsequent attachment of a biotinylated nucleic acid (Holmstrom et al., Anal. Biochem. 209:278-283, 1993). Immobilization can also be carried out by coating a silicon, glass or other substrate with poly-E-Lys (lysine), followed by covalent attachment of either amino- or sulfhydryl-modified nucleic acids using bifunctional crosslinking reagents (Running et al., BioTechniques 8:276-277, 1990; Newton etal., Nucleic Acids Res. 21:1155-62, 1993). Amine residues can be introduced onto a substrate through the use of aminosilane for cross-linking.

Immobilization can take place by direct covalent attachment of 5′-phosphorylated nucleic acids to chemically modified substrates (Rasmussen et al., Anal. Biochem. 198:138-142, 1991). The covalent bond between the nucleic acid and the substrate is formed by condensation with a water-soluble carbodiimide or other cross-linking reagent. This method facilitates a predominantly 5′-attachment of the nucleic acids via their 5′-phosphates. Exemplary modified substrates would include a glass slide or cover slip that has been treated in an acid bath, exposing SiOH groups on the glass (U.S. Pat. No. 5,840,862).

DNA is commonly bound to glass by first silanizing the glass substrate, then activating with carbodiimide or glutaraldehyde. Alternative procedures can use reagents such as 3-glycidoxypropyltrimethoxysilane (GOP), vinyl silane or aminopropyltrimethoxysilane (APTS) with DNA linked via amino linkers incorporated either at the 3′ or 5′ end of the molecule. DNA can be bound directly to membrane substrates using ultraviolet radiation. Other non-limiting examples of immobilization techniques for nucleic acids are disclosed in U.S. Pat. Nos. 5,610,287, 5,776,674 and 6,225,068. Commercially available substrates for nucleic acid binding are available, such as Covalink, Costar, Estapor, Bangs and Dynal. The skilled artisan will realize that the disclosed methods are not limited to immobilization of nucleic acids and are also of potential use, for example, to attach one or both ends of oligonucleotide coded probes to a substrate.

The type of substrate to be used for immobilization of the nucleic acid or other target molecule is not limiting. In various embodiments of the invention, the immobilization substrate can be magnetic beads, non-magnetic beads, a planar substrate or any other conformation of solid substrate comprising almost any material. Non-limiting examples of substrates that can be used include glass, silica, silicate, PDMS (poly dimethyl siloxane), silver or other metal coated substrates, nitrocellulose, nylon, activated quartz, activated glass, polyvinylidene difluoride (PVDF), polystyrene, polyacrylamide, other polymers such as poly(vinyl chloride) or poly(methyl methacrylate), and photopolymers which contain photoreactive species such as nitrenes, carbenes and ketyl radicals capable of forming covalent links with nucleic acid molecules (See U.S. Pat. Nos. 5,405,766 and 5,986,076).

Bifunctional cross-linking reagents can be of use in various embodiments of the invention. The biftnctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3 -(3-dimethylaminopropyl) carbodiimide (EDC).

In certain aspects, the substrate for methods disclosed herein is a biochip. FIG. 3 provides an example of a biochip design and method for sequence determination of a target nucleic acid using a biochip and the methods provided herein. A biochip according to this example contains many probe areas 310. Each probe area 310 (for example, “A” or “B”) has multi-capture probe sets 320 (for example, I and 2). Capture probes 20 in each capture probe set 350 have the same sequence. In other words, capture probes 20 on spots 1A and 1B have the same nucleotide sequence but locate in separate compartment areas. Many Raman probe sets 320 are made (for example, “a” and “b”).

Each Raman-active oligonucleotide probe set 350 is a pool of different Raman-active oligonucleotide probes 40. Each set has more than one oligonucleotide and each oligonucleotide has a unique label. To prepare a Raman-active oligonucleotide probe 40, probe molecules of the same sequence are synthesized and, according to this example, a unique Raman label 45 is attached to each molecule 55 (i.e. the same nucleotide sequence has the same Raman label 45). Many Raman-active oligonucleotide probes 40 with different nucleotide sequences and different Raman labels 45 can be pooled to form a Raman probe set 340. A different set 350 of Raman probes 40 can be formed with different probe sequences 55. However, the same Raman labels 45 can be used in more than one Raman probe sets 320. Therefore, a Raman probe sequence 55 can be identified by its set ID and its Raman label. When multi-copies of a target sequence 10, for example from biological sample without amplification, are fragmented into short and over-lapping segments, they can hybridize to the capture probes 20 on the chip. When Raman probe sets 350 are added separately to the probe areas 310, the Raman probes 40 will hybridize to the target nucleic acid molecules 10. In the presence of ligase, capture probe molecules 20 and Raman probe molecules 40 can be joined, thereby immobilizing the Raman probe molecules 40 in a target sequence-dependent manner. Individual Raman probes 45 can be detected by SERS scanning. Finally, the target sequence is resolved.

Accordingly, in certain aspects, a first population of Raman-active oligonucleotide probes 40 are contacted with probe-target duplex nucleic acids at a first spot of a series of spots 340, and a second population of Raman-active oligonucleotide probes 40 are contacted with the probe-target duplex nucleic acids at a second spot 340 of the series of spots, wherein the first population of Raman-active oligonucleotide probes 40 and the second population of Raman-active oligonucleotide probes 40 include at least one different oligonucleotide probe. Furthermore, the first population of Raman-active oligonucleotide probes 40 and the second population of Raman-active oligonucleotide probes 40 include at least one Raman-active oligonucleotide probe 40 with an identical Raman label 45 but a different oligonucleotide 55 to which the label 45 is bound. Furthermore, in certain aspects, each spot location can include a different capture oligonucleotide probe 20. In addition, the series of spot locations include spot locations with identical capture oligonucleotide probes 20 and spot locations with different capture oligonucleotide probes 20.

In another embodiment, a detection system is provided that includes a Raman spectrometer with a light source, a Raman active surface in optical communication with the light source, and a population of Raman-active oligonucleotide probes that include an undetectable oligonucleotide associated with a positively charged enhancer, wherein the Raman-active oligonucleotide probes are deposited on the Raman active surface. As indicated herein, the positively charged enhancer can be, for example, an amine group enhancer. The system is used to perform methods of sequencing by hybridization using Raman-labeled oligonucleotide probes, as discussed.

As will be understood, sequencing data generated using methods disclosed herein can be used for biomedical research and clinical diagnosis. For example, the methods disclosed herein can be used to generate sequence information for large scale genome sequencing, sequence comparison, genotyping, disease correlation, drug development, pathogen detection, and genetic screening. For example, methods disclosed herein can be used to determine primary sequence information of a subject, such as a sequence a single nucleotide polymorphism, a fragment of a gene, or a complete gene that is related to a disease. Therefore, the methods can be used to diagnose the disease or to provide prognostic information.

In certain aspects, a target molecule is isolated from a biological sample before it is detected by the methods of the present invention. For example, the biological sample can be from a mammalian subject, for example a human subject. The biological sample can be virtually any biological sample, particularly a sample that contains RNA or DNA from a subject. The biological sample is, for example, urine, blood, plasma, serum, saliva, semen, stool, sputum, cerebral spinal fluid, tears, mucus, and the like. The biological sample can be a tissue sample which contains, for example, 1 to 10,000,000; 1000 to 10,000,000; or 1,000,000 to 10,000,000 somatic cells. The sample need not contain intact cells, as long as it contains sufficient RNA or DNA for the methods of the present invention, which in some aspects require only I molecule of RNA or DNA. According to aspects of the present invention wherein the biological sample is from a mammalian subject, the biological or tissue sample can be from any tissue. For example, the tissue can be obtained by surgery, biopsy, swab, stool, or other collection method.

In other aspects, the biological sample contains a pathogen, for example a virus or a bacterial pathogen. In certain aspects, the template nucleic acid is purified from the biological sample before it is contacted with a probe, however. The isolated template nucleic acid can be contacted with a reaction mixture without being amplified.

As used herein, “about” means within ten percent of a value. For example, “about 100” would mean a value between 90 and 110.

“Nucleic acid” encompasses DNA, RNA (ribonucleic acid), single-stranded, double-stranded or triple stranded and any chemical modifications thereof. Virtually any modification of the nucleic acid is contemplated. A “nucleic acid” can be of almost any length, from oligonucleotides of 2 or more bases up to a full-length chromosomal DNA molecule. Nucleic acids include, but are not limited to, oligonucleotides and polynucleotides. A “polynucleotide” as used herein, is a nucleic acid that includes at least 25 nucleotides.

Polymorphisms are allelic variants that occur in a population. A polymorphism can be a single nucleotide difference present at a locus, or can be an insertion or deletion of one or a few nucleotides. As such, a single nucleotide polymorphism (SNP) is characterized by the presence in a population of one or two, three or four nucleotide occurrences (i.e., adenosine, cytosine, guanosine or thymidine) at a particular locus in a genome such as the human genome. As indicated herein, methods disclosed herein provide for the detection of a nucleotide occurrence at a SNP location or a detection of both genomic nucleotide occurrences at a SNP location for a diploid organism such as a mammal. Furthermore, methods disclosed herein provide for the detection of more than 1 SNP in a single reaction, for example, 2, 3, 4, 5, 6, 7, 8, 9, 10, 11, 12, 13, 14, 15, 16, 17, 18,19, 20, 25, 50, 100, or more SNPs.

A “target” or “analyte” molecule is any molecule that can bind to a labeled probe, including but not limited to nucleic acids, proteins, lipids and polysaccharides. In some aspects of methods, binding of a labeled probe to a target molecule can be used to detect the presence of the target molecule in a sample.

Nucleic acid molecules to be sequenced using methods provided herein can he prepared by any technique known in the art. In certain embodiments of the invention, the nucleic acids are naturally occurring DNA or RNA molecules. Virtually any naturally occurring nucleic acid can be sequenced by the disclosed methods including, without limit, chromosomal, mitochondrial and chloroplast DNA and ribosomal, transfer, heterogeneous nuclear and messenger RNA. In some embodiments, the nucleic acids to be analyzed can be present in crude homogenates or extracts of cells, tissues or organs. In other embodiments, the nucleic acids can be partially or fully purified before analysis. In alternative embodiments, the nucleic acid molecules to be analyzed can be prepared by chemical synthesis or by a wide variety of nucleic acid amplification, replication and/or synthetic methods known in the art.

Methods of the present invention analyze nucleic acids that in some aspects are isolated from a cell. Methods for purifying various forms of cellular nucleic acids are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). The methods disclosed in the cited references are exemplary only and any variation known in the art can be used. In cases where single stranded DNA (ssDNA) is to be analyzed, ssDNA can be prepared from double stranded DNA (dsDNA) by any known method. Such methods can involve heating dsDNA and allowing the strands to separate, or can alternatively involve preparation of ssDNA from dsDNA by known amplification or replication methods, such as cloning into M13. Any such known method can be used to prepare ssDNA or ssRNA.

Although certain embodiments of the invention concern analysis of naturally occurring nucleic acids, such as polynucleotides, virtually any type of nucleic acid could be used. For example, nucleic acids prepared by various amplification techniques, such as polymerase chain reaction (PCR3) amplification, could be analyzed. (See U.S. Pat. Nos. 4,683,195, 4,683,202 and 4,800,159.) Nucleic acids to be analyzed can alternatively be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts can be isolated from vector DNA, for example, by excision with appropriate restriction endonucleases, followed by agarose gel electrophoresis. Methods for isolation of nucleic acid inserts are known in the art. The disclosed methods are not limited as to the source of the nucleic acid to be analyzed and any type of nucleic acid, including prokaryotic, bacterial, viral, eukaryotic, mammalian and/or human can be analyzed within the scope of the claimed subject matter.

In various embodiments of the invention, multiple copies of a nucleic acid can be analyzed by labeled oligonucleotide probe hybridization, as discussed below. Preparation of single nucleic acids and formation of multiple copies, for example by various amplification and/or replication methods, are known in the art. Alternatively, a single clone, such as a BAC, YAC, plasmid, virus, or other vector that contains a single nucleic acid insert can be isolated, grown up and the insert removed and purified for analysis. Methods for cloning and obtaining purified nucleic acid inserts are well known in the art.

In various embodiments of the invention, hybridization of a target nucleic acid to a population of labeled oligonucleotides can be performed under stringent conditions that only allow hybridization between fully complementary nucleic acid sequences. Low stringency hybridization is generally performed at 0.15 M to 0.9 M NaCl at a temperature range of 20° C. to 50° C. High stringency hybridization is generally performed at 0.02 M to 0.15 M NaCl at a temperature range of 50° C. to 70° C. It is understood that the temperature and/or ionic strength of an appropriate stringency are determined in part by the length of an oligonucleotide probe, the base content of the target sequences, and the presence of formamide, tetramethylammonium chloride or other solvents in the hybridization mixture. The ranges mentioned above are exemplary and the appropriate stringency for a particular hybridization reaction is often determined empirically by comparison to positive and/or negative controls. The person of ordinary skill in the art is able to routinely adjust hybridization conditions to allow for only stringent hybridization between exactly complementary nucleic acid sequences to occur.

It is unlikely that a given target nucleic acid will hybridize to contiguous probe sequences that completely cover the target sequence. Rather, multiple copies of a target can be hybridized to pools of labeled oligonucleotides and partial sequence data collected from each. The partial sequences can be compiled into a complete target nucleic acid sequence using publicly available shotgun sequence compilation programs.

In certain embodiments of the invention, labeled oligonucleotides are detected while still attached to a target molecule. Given the relatively weak strength of the binding interaction between short oligonucleotide probes and target nucleic acids, such methods can be more appropriate where, for example, labeled probes have been covalently attached to the target molecule using cross-linking reagents.

In various embodiments of the invention, oligonucleotide probes can be DNA, RNA, or any analog thereof, such as peptide nucleic acid (PNA), which can be used to identify a specific complementary sequence in a nucleic acid. In certain embodiments of the invention one or more oligonucleotide probe libraries can be prepared for hybridization to one or more nucleic acid molecules. For example, a set of labeled oligonucleotide probes containing all 4096 or about 2000 non-complementary 6-mers, or all 16,384 or about 8,000 non-complementary 7-mers can be used. If non-complementary subsets of oligonucleotide probes are to be used, a plurality of hybridizations and sequence analyses can be carried out and the results of the analyses merged into a single data set by computational methods. For example, if a library comprising only non-complementary 6-mers were used for hybridization and sequence analysis, a second hybridization and analysis using the same target nucleic acid molecule hybridized to those labeled probe sequences excluded from the first library can be performed.

Oligonucleotides of a population of labeled oligonucleotide can be prepared by any known method, such as by synthesis on an Applied Biosystems 381A DNA synthesizer (Foster City, Calif.) or similar instruments. Alternatively, oligonucleotides can be purchased from a variety of vendors (e.g., Proligo, Boulder, Colo.; Midland Certified Reagents, Midland, Tex.). In embodiments where oligonucleotides are chemically synthesized, the signal molecules, such as a Raman label, or positively-charged enhancer, can be covalently attached to one or more of the nucleotide precursors used for synthesis. Alternatively, the signal molecules, can be attached after the oligonucleotide probe has been synthesized. In other alternatives, the Raman labels can be attached concurrently with oligonucleotide synthesis.

In certain aspects of the invention, labeled oligonucleotide probes include peptide nucleic acids (PNAs). PNAs are a polyamide type of DNA analog with monomeric units for adenine, guanine, thymine, and cytosine. PNAs are commercially available from companies such as PE Biosystems (Foster City, Calif.). Alternatively, PNA synthesis can be performed with 9-fluoroenylmethoxycarbonyl (Fmoc) monomer activation and coupling using 0-(7-azabenzotriazol- 1-yl)-1,1,3,3-tetramethyluronium hexafluorophosphate (HATU) in the presence of a tertiary amine, N,N-diisopropylethylamine (DIEA). PNAs can be purified by reverse phase high performance liquid chromatography (RP-HPLC) and verified by matrix assisted laser desorption ionization—time of flight (MALDI-TOF) mass spectrometry analysis.

Further provided herein is a kit for performing the above methods. The kit includes a population of Raman-active oligonucleotide probes, wherein one or more of the Raman-active oligonucleotide probes is bound to a positively-charged enhancer. The kit can also include a substrate with a population of bound capture oligonucleotide probes, as discussed herein. In certain examples, the kit further includes a silver colloid or nanoparticle. In addition, the kit can include a Raman active surface.

The following embodiments are based on the discovery of a sensitive and simple detection method that is able to distinguish target molecules with subtle differences. The method is important for biomedical applications, such as single nucleotide polymorphism detection in genotyping, as discussed above. The method is efficient in that chemical modifications and lengthy sample preparation techniques are not required, and a large number of targets can be assayed in a single device in a short time. In addition, the method is relatively low cost, since very small amounts of sample and reagents are required.

Not to be limited by theory, the disclosed methods are based in part on the concept that there are configuration differences in labels that are attached to a probe when the same probe forms complexes with two targets that differ by a subtle structural difference, and that these steric configuration differences can be resolved by optical techniques. The data provided herein suggest that a single nucleic acid probe can detect the differences among perfect matched and mismatched targets using fluorescent methods.

Furthermore, in certain aspects the method for detecting subtle structural differences is performed using a Raman detection methods. These aspects rely on the fact that Raman detection methods provide more structural information than fluorescent detection methods. Furthermore, SERS Raman has the potential of single molecule detection sensitivity. Finally, a microfluidic MEMS device can be used to separate a sample into individual probe-target complexes in small liquid cavities, which can be detected by SERS.

Accordingly, in another embodiment, a method for detecting a target structure in a first specific binding pair member is provided, that includes contacting the first binding pair member with a second specific binding pair member, wherein the second specific binding pair member binds to the first specific binding pair member, and wherein the second specific binding pair member comprises a first label and a second label, wherein the first label is capable of affecting the optical signal of the second label in a manner that is affected by the target structure of the first binding pair member upon binding of the first binding pair member to the second binding pair member. The signal from the second label is detected and used to determine the target structure of the first binding pair member.

The first specific binding pair member in this embodiment is a target molecule and the second specific binding pair member is a probe. A probe is a molecular polymer (i.e., protein, nucleic acid, etc.) that recognizes and binds specifically to its ligand (i.e. the target molecule). The probe molecule is a specific binding pair member, for example, a nucleic acid, such as an oligonucleotide or a polynucleotide; a protein or peptide fragment thereof, such as a receptor or a transcription factor, an antibody or an antibody fragment, for example, a genetically engineered antibody, a single chain antibody, or a humanized antibody; a lectin; a substrate; an inhibitor; an activator; a ligand; a hormone; a cytokine; a chemokine; and/or a pharmaceutical. When a target molecule is a protein, for example, the probe is a protein (antibody); when a target is nucleic acid, the probe is typically a nucleic acid such as an oligonucleotide.

As used herein, the term “specific binding pair member” refers to a molecule that specifically binds or selectively hybridizes to another member of a specific binding pair. Specific binding pair member include, for example, an oligonucleotide and a nucleic acid to which the oligonucleotide selectively hybridizes, or a protein and an antibody that binds to the protein. As used herein, the term “selective hybridization” or “selectively hybridize” refers to hybridization under highly stringent physiological conditions. The term “binds specifically” or “specific binding activity,” when used in reference to an antibody means that an interaction of the antibody and a particular epitope has a dissociation constant of at least about 1×10−6, generally at least about 1×10−7, usually at least about 1×10−8, and particularly at least about 1×10−9 or 1×10−10 or less.

In a related embodiment, a method is provided for determining a nucleotide occurrence at a target nucleotide position of a target nucleic acid, that includes contacting the target nucleic acid with a labeled oligonucleotide probe that binds to the target nucleic acid, wherein the labeled oligonucleotide probe includes a first label and a second label, the first label being capable of affecting an optical property of the second label, to form a probe-target complex, and detecting an optical property of the probe-target complex. The nucleotide occurrence at the target nucleotide position affects the orientation of the first label and the second label, thereby affecting an optical property of the second label. The optical property is, for example, a fluorescent signal or a Raman signal generated by the second label. Properties of the generated signal allow a determination of the nucleotide occurrence at the target nucleotide position.

In certain aspects, an alternating current (AC) is applied to the probe-target complex before it is detected to enhance the difference in the affect of the first probe on the second probe fluorescent signal or Raman signal depending on whether the target polynucleotide and the labeled probe comprise complementary nucleotides at the target nucleotide position. The AC voltage that is applied can be, for example, 10-100 mV with AC frequency of 1 Hz to 1 MHz.

The fluorescent signal and the Raman signal, for example are fluorescent spectra or Raman spectra, respectively. Methods for detecting fluorescent signals and Raman signals, some of which are provided herein, are well known in the art. In certain aspects, the first label and the second label are a FRET pair, as discussed in further detail herein. In one example, the FRET pair is TAMRA and ROX.

Accordingly, labels are optically detectable moieties that are attached to a probe. A label can be a fluorescence dye, Raman label or any small molecule that can be recognized by an optical technique. Examples of Raman labels are provided hererinabove.

The first label and the second label of a probe are selected to form a donor/acceptor pair comprising a fluorescent or Raman donor and a fluorescent or Raman acceptor capable of fluorescence resonance energy transfer or Raman energy transfer with each other in response to activation of the fluorescent or Raman donor by light of a predetermined wavelength or band of wavelengths.

The excitation and emission spectra of a fluorescent or Raman label and the label to which it is paired determines whether it is a fluorescent or Raman donor or a fluorescent or Raman acceptor. Examples of molecules that are used in FRET include the dye fluorescein and fluorescein derivatives such as 5-carboxyfluorescein (5-FAM), 6-carboxyfluorescein (6-FAM), fluorescein-5-isothiocyanate (FITC), 2′7′-dimethoxy-4′5′-dichloro-6-carboxyfluorescein (JOE); rhodamine and rhodamine derivatives such as N,N,N′,N′-tetramethyl-6-carboxyrhodamine (TAMRA), 6-carboxyrhodamine (R6G), tetramethyl-indocarbocyanine (Cy3), tetramethyl-benzindocarbocyanine (Cy3.5), tetramethyl-indodicarbocyanine (Cy5), tetramethyl-indotricarbocyanine (Cy7), 6-carboxy-X-rhodamine (ROX); hexachloro fluorescein (HEX), tetrachloro fluorescein TET; R-Phycoerythrin, 4-(4′-dimethylaminophenylazo) benzoic acid (DABCYL), and 5-(2′-aminoethyl)aminonaphthalene-1-sulfonic acid (EDANS). Table 1 lists exemplary donor and acceptor donor and acceptor pairs, including the maximum absorbance (Abs) and emission (Em) for each. The term fluorescent acceptor encompasses fluorescence quenchers. Exemplary quencher dyes are well known in the art, e.g. as described by Clegg, “Fluorescence resonance energy transfer and nucleic acids,” Methods of Enzymology, 211:353-389 (1992).

TABLE I
Exemplary FRET pairs

| | Abs/Em | | Abs/Em |
|---|---|---|---|
| Donor | Max | Acceptor | Max |
| Fluorescein and | 495/520 | Tetramethyl Rhodamine | 555/580 |
| derivatives (FITC, | | derivatives (TRITC, | |
| 5-FAM, 6-FAM, | | TAMRA, etc.) | |
| etc.) | | | |
| TET | 521/536 | TAMRA | 555/580 |
| HEX | 535/556 | TAMRA | 555/580 |
| Cy³ | 552/570 | Cy⁵ | 649/670 |
| Cy³ | 552/570 | Cy^{3.5} | 581/596 |
| Cy^{3.5} | 581/596 | Cy⁵ | 649/670 |
| R-Phycoerythrin | 546/578 | Cy⁵ | 649/670 |
| Cy⁵ | 649/670 | Cy⁷ | 743/767 |

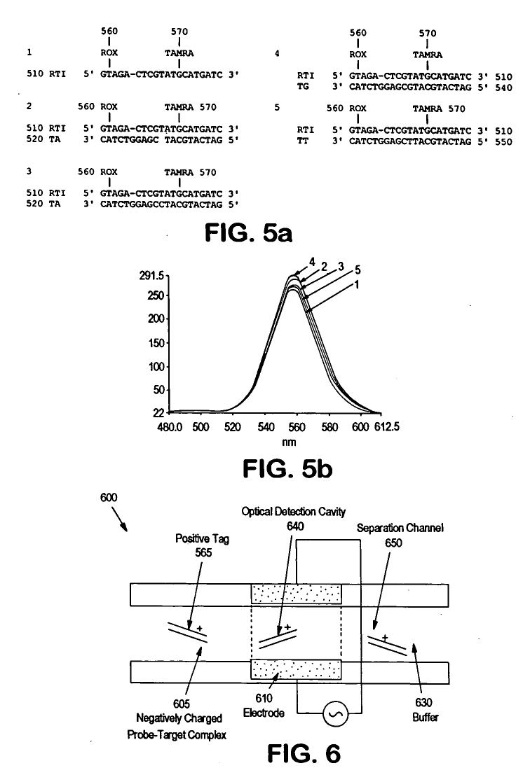

The methods discussed in the following paragraphs can be used to attach Raman or fluorescent labels to oligonucleotides for use in any embodiment disclosed herein. The fluorescent or Raman labels can be incorporated or attached to the 5′-end nucleotide of an oligonucleotide probe, to the 3′-end nucleotide of an oligonucleotide probe, and to non-terminal or internal nucleotides of the oligonucleotide probes. In the embodiment of FIG. 5, fluorescent moieties are attached to internal nucleotides.

With 5′-end labeled oligonucleotides, the fluorescent or Raman labels (i.e. moieties) are typically attached to 5′-end nucleotide of the oligonucleotide probe prior to synthesis and the labeled nucleotide is incorporated into the oligonucleotide synthesis following known procedures (e.g., Yang and Millar in Methods in Enzymology, Vol. 278, pages 417-444, (1997)).

For internal labeling, amino-modified bases are typically introduced into the oligonucleotide during synthesis using Amino-Modifier C6 dT (e.g. available from Glen Research). Active-ester derivatives of the label are then coupled to the amino-modified base post-synthetically. This is advantageous because the reagents, such as cyanine-dye-labeled nucleotides, are readily available and the label does not interfere with the hybridization of the probe.

Alternative methods for attachment of fluorophores or Raman labels to oligonucleotides are described in Khanna et al, U.S. Pat. No. 4,351,760; Marshall; Mechnen et at, U.S. Pat. No. 5,188,934; Woo et al, U.S. Pat. No. 5,231,191; and Hobbs, Jr. U.S. Pat. No. 4,997, 928).

In methods of this embodiment of the invention, the labeled oligonucleotide probe and the target nucleic acid are hybridized under conditions that result in a hybridization complex in which the fluorescent or Raman donor and fluorescent or Raman acceptor are spaced apart from each other a distance such that they are capable of fluorescence resonance energy transfer or Raman scattering energy transfer in response to activation of the fluorescent or Raman donor by light of a predetermined wavelength. Activation and detection of the fluorescent or Raman donor and the fluorescent or Raman acceptor can be performed at discrete wavelength of light or band of wavelengths such as through a filter.

The efficiency of fluorescence or resonance energy transfer has been reported to be proportional to D×10⁻⁶, where D is the distance between the donor and acceptor (Forster, Z. Naturforsch A, 1949, 4:321-327). Accordingly, fluorescence resonance energy transfer typically occurs at distances of between 10-70 Angstroms, in some cases 30-60 Angstroms. The fluorescent donor and fluorescent acceptor are typically separated by between about 5 base-pairs (bp) and about 24 bp in a hybridized probe pair duplex. The separation distance can be less where the fluorescent acceptor is a quencher.

In certain aspects, the fluorescent or Raman donor and fluorescent or Raman acceptor are separated by 8 bp-18 bp on an oligonucleotide probe. For example, the fluorescent or Raman donor and the fluorescent or Raman acceptor are separated by 10 bp-13 bp.

Detection is performed by detecting light emitted by the fluorescent or Raman donor, the fluorescent or Raman acceptor, or both the fluorescent and Raman donor and acceptor (e.g. a ratio). In one embodiment, the fluorescent or Raman donor and the fluorescent or Raman acceptor are both fluorophores. The first fluorophore is activated with light of the appropriate wavelength or band of wavelengths, which is a function of the particular fluorophore. Fluorescence measurements can be made, for example, using a Wallac 1420 Victor₂ multilabel counter, a Perkin Elmer LS50B luminescence spectrometer, or other suitable instruments. In an alternative aspect, the fluorescent donor is a fluorophore and the fluorescent acceptor is a quencher and detection is performed by measuring the emission of the fluorophore. The fluorescent and/or Raman spectra generated by the labeled oligonucleotide probe are different depending on the nucleotide sequence of the target nucleic acid molecule.

The invention can be used to detect more than one nucleotide occurrence at more than one target nucleotide position in one or more samples. The target positions can reside on distinct nucleic acids, or more than one target position can reside on a particular nucleic acid. In one embodiment, the method is performed with more than one oligonucleotide probe pair wherein the donor/acceptor pairs of at least two probes are different and the wavelength or band of wavelengths of light used for activation of the fluorescent or Raman donor of the at least two different probes is distinct. Accordingly in these aspects, a series of nucleotide occurrences for one or more target nucleotides are determined using a population of labeled probes. Accordingly, methods of these aspects provide powerful tools for allowing analysis of a series of SNPs in a biological sample.

In another embodiment, either the first probe or the second probe is immobilized on a solid surface. In this embodiment, it is not required that the donor/acceptor pairs of at least two probe pairs are different and the wavelength of light used for activation of the fluorescent or Raman donor of the at least two different probes is distinct. The identity and specificity of the nucleotide occurrence at each target position is determined by the spatial localization of the probes. Alternatively, the target nucleic acids are immobilized or attached at discrete locations to detect multiple polynucleotide targets with specificity. The detection of nucleotide occurrences at target positions of a target nucleic acid is useful for a variety of applications, including for example, the detection of nucleotide occurrences associated with single nucleotide polymorphisms, insertions, deletions, and multiple mutations.

In various embodiments of the invention, hybridization of a target nucleic acid to a labeled oligonucleotide probe can be performed under a variety of stringency conditions. For example, low stringency hybridization can be performed. Low stringency hybridization is generally performed at 0.15 M to 0.9 M NaCl at a temperature range of 20° C. to 50° C. High stringency hybridization is generally performed at 0.02 M to 0.15 M NaCl at a temperature range of 50° C. to 70° C. It is understood that the temperature and/or ionic strength of an appropriate stringency are determined in part by the length of an oligonucleotide probe, the base content of the target sequences, and the presence of formamide, tetramethylammonium chloride or other solvents in the hybridization mixture. The ranges mentioned above are exemplary and the appropriate stringency for a particular hybridization reaction is often determined empirically by comparison to positive and/or negative controls. The person of ordinary skill in the art is able to routinely adjust hybridization conditions to allow for only stringent hybridization between exactly complementary nucleic acid sequences to occur.

In certain aspects, probe-target complexes are individually passed through an optical detector to read the fluorescent signal or Raman spectra generated by the probe-target complexes. For example, the optical detector can be a MEMS detection device disclosed herein below. Using this device, for example individual probe-target complexes can be individually passed through using a microelectromechanical system having a channel that is sufficiently narrow to allow only one probe-target complex to pass.

FIG. 5 provides an example of using a labeled oligonucleotide probe for detection of a nucleotide occurrence at a target position, as discussed in further detail in the Examples section herein. As illustrated in FIG. 5A, the fluorescent labels Rox 560 and Tamra 570 were attached to a synthetic oligonucleotide sequence (RTI) 510. The labeled oligonucleotide probe is used as a probe (RTI) 510 to detect a single nucleotide difference in target nucleic acids 520 (TA), 530 (TC), 540 (TG), and 550 (TT). The four targets nucleic acids 520, 530, 540, and 550 differ only in the nucleotide position corresponding to the nucleotide bound to TAMRA in the labeled oligonucleotide probe 510 (A, C, G and T, in target nucleic acids 520, 530, 540, and 550 respectively). The labeled oligonucleotide probe 510 and the target nucleic acids 520, 530, 540, and 550 form probe-target complexes in a solution similar to a physiological saline in terms of ionic strength and pH value.

When a probe molecule binds to a target molecule, they form a double-stranded molecule complex. When the base-pairing is perfect (no mismatch in the middle bases, as in RTI 510 +TA 520), the double helix makes 1 complete turn about every 10 base pairs. Since the 2 labels are located approximately 3-6 nm apart, there can be resonance energy transfer when they are properly oriented and excited. Since the efficiency (E) of energy transfer between 2 labels, also referred to as tag moieties, depends on distance (r): E=1/rˆ6 when r is large than the distance for 50% efficiency (4-6 nm). Any mismatches in the middle bases (i.e. the bases as a target nucleotide position) can alter the r value slightly but the E value significantly. This can happen when the labeled RTI oligonucleotide probe 510 binds to target nucleic acids that do not include a complementary nucleotide at the target nucleotide position (i.e., TC 530, TG 540, or TT 550). The E changes can be detected using optical methods, and can be detected in spectra of fluorescence or Raman analyses.

FIG. 5B shows data from fluorescence assays using the probe-target system of FIG. 5A. The differences in the synchronous scan spectra (delta =30 nm) indicate that there are differences in the relative distances between Rox and Tarnra in these probe-target complexes. The differences are most likely caused by mispaired bases.

In theory, more information can be derived from the above system when Raman scattering techniques, including SERS Raman, are used. The relative intensities of Raman peaks can be used as indicators for different sequence structures. The method can be applied to other systems in addition to nucleic acids, provided suitable tagged probes are available.

In another aspect, a spectra database or library is constructed for known genotypes or known molecules. For example, a spectra database or library can be created for nucleotide occurrences at known single nucleotide polymorphisms (SNPs). A Raman spectra or fluorescent spectra generated for a target molecule as discussed above, can be compared to the spectra database or library to identify the nucleotide occurrence at the target nucleotide position of the target polynucleotide.

The method according to these aspects, can then be used for example, to detect the nucleotide occurrence for the SNPs in biological samples. The biological sample is, for example, urine, blood, plasma, serum, saliva, semen, stool, sputum, cerebral spinal fluid, tears, mucus, and the like, as discussed above.

Accordingly, provided herein is a database that includes Raman spectra profiles for a population of labeled oligonucleotide probes that include a first label and a second label, wherein the Raman spectra include a series of groups of Raman spectra, wherein each group of Raman spectra are generated from an identical probe sequence bound to a series of target polynucleotides that include an identical nucleotide sequence except at a target position. In certain aspects, the target position represents a single nucleotide polymorphism (SNP) position.

In certain aspects, probe-target complexes are individually passed through an optical detection device to read the fluorescent signal or Raman spectra generated by the probe-target complexes. Using this device, for example individual probe-target complexes can be individually passed through using a microelectromechanical system having a channel that is sufficiently narrow to allow only one probe-target complex to pass through at a time.

FIG. 6 illustrates a MEMS device 600 for single probe-target complex detection. The device is typically used with the method disclosed herein, for detecting a nucleotide occurrence at a target nucleotide position using a labeled oligonucleotide probe that includes a first label (e.g. FRET donor) and a second label (e.g. FRET acceptor 565), wherein the first label and the second label 565 are a FRET pair. When a sample contains multiple copies of a target nucleic acid, such as a biological sample, probes are added to the sample to form probe-target complexes 605. These complexes 605 are separately passed through an optical detector cavity 640 and individual spectra are recorded. The data, for example, can then be searched against a data library. Information is analyzed statistically. For example, the library can include spectra for all known nucleotide occurrences at a target position of a target nucleic acid using the labeled oligonucleotide probe.

To achieve individual complex separation, a MEMS device 600 can be used. A sample is allowed to pass a narrow separation channel 650 so that statistically only one complex 605 occupies a detection cavity 640. To enhance the difference between perfectly hybridized complex and complex with a mismatch, an alternating current (AC) field is generated using an AC power source 620 with adjustable frequency and voltage, to induce a conformation change in the mismatched complex that is relatively more flexible than the perfectly matched complex. These embodiments utilizing an AC field are typically performed in aspects of the invention that utilize fluorescent detection methods. Not to be limited by theory, the change in the relative distance between different labels can affect the electron distribution in the complex or alter the energy transfer efficiency between light sensitive labels, which can result in more distinguishable signal.

Accordingly, the present invention provides a microelectromechanical system (MEMS) device for detecting a target molecule, that includes a sample inlet for accepting a sample that includes a complex of a biomolecule and a labeled binding pair member comprising a first label and a second label, wherein the first label is capable of affecting a fluorescent signal or a Raman spectra of the second label, a separation channel 650 fluidly connected to the sample inlet, and an optical detection means and an optical detection cavity 640.

The detection means is typically a fluorescent detection means or a Raman detection means, both of which are well known in the art. In certain aspects, Raman labels are used and the detection means is a Raman detection system.

In certain aspects, where a fluorescence detection means is employed and fluorescent labels are employed, the device can further include an electrode 610, and an alternating current power source 620. The power source 620 can be used to apply an AC current to a probe-target complex, as disclosed herein. Therefore, the alternating current power source 620 can generate an alternating current field in the optical detection cavity 640.

In certain aspects, wherein a Raman detection means is employed, the separation channel is sufficiently narrow such that statistically only one probe-target complex occupies a detection cavity at a time. For example, the channel can be in the range of 0.5 to 10 microns, since for optical detection the laser spot size is normally larger than 0.3 micron, typically 1 to 5 microns. In these aspects, the optical assembly further comprises a light source for Raman spectroscopy, such as an Ar-ion laser.

MEMS are integrated systems including mechanical elements, sensors, actuators, and electronics. All of those components can be manufactured by microfabrication techniques on a common chip, of a silicon-based or equivalent substrate (e.g., Voldman et al., Ann. Rev. Biomed. Eng 1:401-425, 1999). The sensor components of MEMS can be used to measure mechanical, thermal, biological, chemical, optical and/or magnetic phenomena to detect labels. The electronics can process the information from the sensors and control actuator components such pumps, valves, heaters, etc. thereby controlling the function of the MEMS.

The electronic components of MEMS can be fabricated using integrated circuit (IC) processes (e.g., CMOS or Bipolar processes). They can be patterned using photolithographic and etching methods for computer chip manufacture. The micromechanical components can be fabricated using compatible “micromachining” processes that selectively etch away parts of the silicon wafer or add new structural layers to form the mechanical and/or electromechanical components.

Basic techniques in MEMS manufacture include depositing thin films of material on a substrate, applying a patterned mask on top of the films by some lithographic methods, and selectively etching the films. A thin film can be in the range of a few nanometers to 100 micrometers. Deposition techniques of use can include chemical procedures such as chemical vapor deposition (CVD), electrodeposition, epitaxy and thermal oxidation and physical procedures like physical vapor deposition (PVD) and casting. Methods for manufacture of nanoelectromechanical systems can also be used (See, e.g., Craighead, Science 290:1532-36, 2000.)

In some embodiments, apparatus and/or detectors can be connected to various fluid filled compartments, for example microfluidic channels or nanochannels. These and other components of the apparatus can be formed as a single unit, for example in the form of a chip (e.g. semiconductor chips) and/or microcapillary or microfluidic chips. Alternatively, individual components can be separately fabricated and attached together. Any materials known for use in such chips can be used in the disclosed apparatus, for example silicon, silicon dioxide, polydimethyl siloxane (PDMS), polymethylmethacrylate (PMMA), plastic, glass, quartz, etc.

Techniques for batch fabrication of chips are well known in computer chip manufacture and/or microcapillary chip manufacture. Such chips can be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding, dry etching of silicon dioxide; and electron beam lithography. Methods for manufacture of nanoelectromechanical systems can be used for certain embodiments. (See, e.g., Craighead, Science 290:1532-36, 2000.) Various forms of microfabricated chips are commercially available from, e.g., Caliper Technologies Inc. (Mountain View, Calif.) and ACLARA BioSciences Inc. (Mountain View, Calif.).

In certain embodiments, part or all of the apparatus can be selected to be transparent to electromagnetic radiation at the excitation and emission frequencies used for Raman spectroscopy or fluorescence detection. Suitable components can be fabricated from materials such as glass, silicon, quartz or any other optically clear material. For fluid-filled compartments that can be exposed to various analytes, for example, nucleic acids, proteins and the like, the surfaces exposed to such molecules can be modified by coating, for example to transform a surface from a hydrophobic to a hydrophilic surface and/or to decrease adsorption of molecules to a surface. Surface modification of common chip materials such as glass, silicon, quartz and/or PDMS is known (e.g., U.S. Pat. No. 6,263,286). Such modifications can include, for example, coating with commercially available capillary coatings (Supelco, Bellafonte, PA), silanes with various functional (e.g. polyethyleneoxide or acrylamide, etc).

In certain embodiments, such MEMS apparatus can be use to prepare labeled probes, to separate formed labeled probes from unincorporated components, to expose labeled probes to targets, and/or to detect labeled probes bound to targets.

The invention further includes a kit for performing assays for determining the nucleotide occurrence at a target position of a target nucleic acid. The kit includes a first oligonucleotide probe labeled with a first and second fluorescent or Raman label as disclosed hereinabove. The first probe is substantially or perfectly complementary to the target nucleic acid. The first and second fluorescent or Raman label (i.e. donor or acceptor) are a donor/acceptor pair capable of fluorescence resonance energy transfer or Raman energy transfer with each other in response to activation the donor by light of a predetermined wavelength or band of wavelengths. The kit can further include a silver colloid or nanoparticle. In addition, the kit can include a Raman active surface.

In certain embodiments of the invention, a system for carrying out the methods of any embodiment of the invention can include an information processing and control system. The embodiments are not limiting for the type of information processing system used. Such a system can be used to analyze data obtained from a Raman spectrometer detection system or a fluorescence detection system. An exemplary information processing system can incorporate a computer comprising a bus for communicating information and a processor for processing information. In one embodiment, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor can be a Celeron®, an Itanium®, an X-Scale® or a Pentium Xeone processor (Intel Corp., Santa Clara, Calif.). In various other embodiments of the invention, the processor can be based on Intel® architecture, such as Intel® IA-32 or Intel® IA-64 architecture. Alternatively, other processors can be used.

The computer can further comprise a random access memory (RAM) or other dynamic storage device, a read only memory (ROM) or other static storage and a data storage device such as a magnetic disk or optical disc and its corresponding drive. The information processing system can also comprise other peripheral devices known in the art, such a display device (e.g., cathode ray tube or Liquid Crystal Display), an alphanumeric input device (e.g., keyboard), a cursor control device (e.g., mouse, trackball, or cursor direction keys) and a communication device (e.g., modem, network interface card, or interface device used for coupling to Ethernet, token ring, or other types of networks).

In particular embodiments of the invention, a Raman spectrometer detection system or a fluorescence detection system is connected to the information processing system. Data from the Raman spectrometer or fluorescence detector can be processed by the processor and data stored in the main memory. The processor can analyze the data from the Raman spectrometer or fluorescence detector to identify and/or determine the sequences of labeled probes attached to a surface. By aligning sequences of overlapping labeled probes, the computer can compile a sequence of a target nucleic acid.

In certain embodiments of the invention, custom designed software packages can be used to analyze the data obtained from a detection technique. In alternative embodiments of the invention, data analysis can be performed using an information processing system and publicly available software packages. Non-limiting examples of available software for DNA sequence analysis include the PRISM3 DNA Sequencing Analysis Software (Applied Biosystems, Foster City, Calif.), the Sequencher3 package (Gene Codes, Ann Arbor, Mich.), and a variety of software packages available through the National Biotechnology Information Facility on the worldwide web at nbif.org/links/1.4.1.php.

Apparatus for labeled probe preparation, use and/or detection can be incorporated into a larger apparatus and/or system. In certain embodiments, the apparatus can include a micro-electro-mechanical system (MEMS) as disclosed above.

The following examples are intended to illustrate but not limit the invention.

### EXAMPLE 1

### Enhancement of Raman Intensity of an Oligonucleotide by Addition of an Amine Group

This example illustrates the increased intensity provided by binding an amine group to an oligonucleotide. Oligonucleotides were custom synthesized and HPLC purified by Qiagen-Operon (Alameda, Calif.). An amino group was added to either a 3′terminus, a 5′ terminus, or both a 3′ and a 5′ terminus during or after oligo synthesis using techniques known in art. Raman signals were detected by Raman spectroscopy using 514 nm light generated by an argon ion laser.

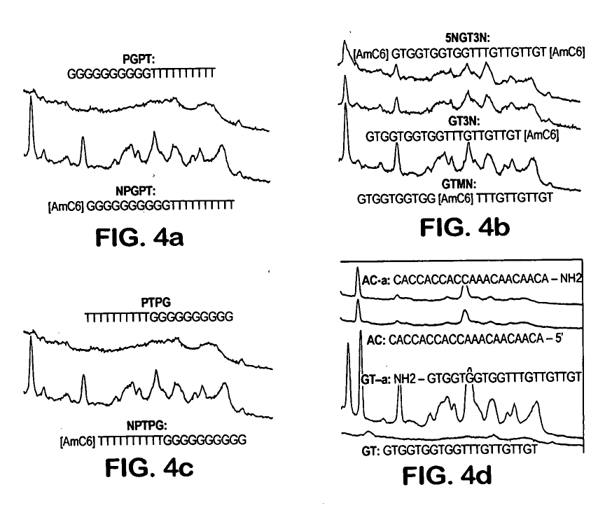

As illustrated in FIGS. 4A and 4C, the intensity of a Raman spectrum produced by an oligonucleotide that includes only guanidine repeats followed by thymidine repeats was increased by additional of a primary amino group with a 6-carbon alkyl chain to the end of an oligonucleotide. A similar enhancement was seen with the addition of a primary amino group with a 6-carbon alkyl chain, as illustrated in FIG. 4B, in which guanosine and thymidine residues were more randomly ordered in the sequence and where one or two primary amino groups were bound to the end of, or bound within, a probe oligonucleotide. The enhancement by the primary amine was not as apparent for oligonucleotides which include purine nucleotides (FIG. 4D). Accordingly, this example illustrates that a positively charged enhancer increases the Raman signal of oligonucleotides that include pyrimidine residues.

### EXAMPLE 2

### Detection of Single Nucleotide Mismatch Using Fluorescent Pairs

This example illustrates a method in which a fluorescent pair is used to detect a single nucleotide mismatch between a target nucleic acid and an oligonucleotide probe. Oligonucleotides were synthesized, HPLC purified, and labeled with ROX or TAMRA by Qiagen-Operon using known methods. Synchronous fluorescent scans were performed using an LS55 (Perkin Elmer). The hybridization was performned under conditions similar to physiological conditions in terms of ionic strength and pH. More specifically, the hybridization was performed in a hybridization reaction mixture that included 100 mM NaCL, 10 mM TrisHCI, and 1 mM EDTA at 22° C.

FIG. 5 provides an example of using a labeled oligonucleotide probe for detection of a nucleotide occurrence at a target position, as disclosed herein. As illustrated in FIG. 5A, the fluorescent labels Rox 560 and Tamra 570 were attached to a synthetic oligonucleotide sequence (RTI) 510. The labeled oligonucleotide probe was used as a probe (RTI) 510 to detect a single nucleotide difference in target nucleic acids 520 (TA), 530 (TC), 540 (TG), and 550 (TT). The four targets nucleic acids 520, 530, 540, and 550 differ only in the nucleotide position corresponding to the nucleotide bound to TAMRA in the labeled oligonucleotide probe 510 (A, C, G and T, in target nucleic acids 520, 530, 540, and 550 respectively). The labeled oligonucleotide probe 510 and the target nucleic acids 520, 530, 540, and 550 form probe-target complexes in a solution similar to a physiological saline in terms of ionic strength and pH value.

FIG. 5B shows data from fluorescence assays using the probe-target system of FIG. 5A. The differences in the synchronous scan spectra (delta=30 nm) indicate that there are differences in the relative distances between Rox and Tamra in these probe-target complexes. The differences are most likely caused by mispaired bases.

Although the invention has been described with reference to the above examples, it will be understood that modifications and variations are encompassed within the spirit and scope of the invention. Accordingly, the invention is limited only by the following claims.

What is claimed is:

1. A method for detecting a nucleic acid, comprising:
a) irradiating the nucleic acid with light, wherein the nucleic acid comprises a positively-charged enhancer; and
b) detecting a Raman signal generated by the irradiated nucleic acid.

2. The method of claim 1, wherein the positively charged enhancer is an amine group.

3. The method of claim 1, wherein the nucleic acid does not generate a detectable signal without the positively-charged enhancer.

4. The method of claim 1, wherein the nucleic acid consists of pyrimidine residues.


SEQUENCE LISTING (9 sequences)

SEQ ID NO: 1 - DNA, 20 bp, Artificial sequence
  Probe
ggggggggggtttttttttt

SEQ ID NO: 2 - DNA, 20 bp, Artificial sequence
  Probe
ttttttttttgggggggggg

SEQ ID NO: 3 - DNA, 21 bp, Artificial sequence
  Probe
gtggtggtggtttgttgttgt

SEQ ID NO: 4 - DNA, 21 bp, Artificial sequence
  Probe
acaacaacaaaccaccaccac

SEQ ID NO: 5 - DNA, 20 bp, Artificial sequence
  Probe
gtagactcgtatgcatgatc

SEQ ID NO: 6 - DNA, 20 bp, Artificial sequence
  Probe
gatcatgcatcgaggtctac

SEQ ID NO: 7 - DNA, 21 bp, Artificial sequence
  Probe
gatcatgcatccgaggtctac

SEQ ID NO: 8 - DNA, 21 bp, Artificial sequence
  Probe
gatcatgcatgcgaggtctac

SEQ ID NO: 9 - DNA, 21 bp, Artificial sequence
  Probe
gatcatgcattcgaggtctac